(19) United States
(12) Patent Application Publication
(10) Pub. No.: US 2016/0338792 A1
(43) Pub. Date: Nov. 24, 2016

(54) Extra-Hospital Medical Workstation

(71) Applicants: Sauel MERCIER, Ville d'Avray (FR); INNOVATIVE MEDICAL PAETNER, Vannes (FR)

(72) Inventor: Samuel Mercier, Ville D'Avray (FR)

(21) Appl. No.: 15/111,311

(22) PCT Filed: Jan. 16, 2015
(86) PCT No.: PCT/EP2015/050807

§ 371 Date: Jul. 13, 2016

(30) Foreign Application Priority Data
Jan. 17, 2014 (FR) 1400095

(51) Int. Cl. A61B 50/15; A61B 50/18; F16M 11/28; A61B 50/30; A61M 5/14; F16M 11/24; A61B 50/33; A61B 90/30

(52) U.S. Cl. CPC A61B 50/15; A61B 50/33; A61B 50/18; A61B 90/30; A61B 50/3001; A61M 5/1417; F16M 11/245; F16M 11/28; A61B 2050/185; A61B 2050/3011

(57) ABSTRACT

The invention relates to a medical equipment structure intended to be used chiefly by medical or paramedical personnel in an extra-hospital environment, comprising at least a work tray (500), as well as a raised hook-up system and attachment means allowing care waste storage accessories to be attached, characterized in that said structure further comprises a platform (10) having a top surface, a bottom surface, and several lateral surfaces, the platform (10) being connected at its bottom surface to folding legs and, by a pivot means, to at least one work tray (500), in that said platform (10) has a mast (300) passing through it, and in that it comprises a means (600) for holding, all of this allowing the collection of these various elements to be transported and set up quickly, stably and level both on flat ground and on uneven ground.

The present invention relates to the technical sector of medical apparatus and equipment, the latter being mainly designed for use outside of hospitals.

To better understand the problems of medical work in the environment outside hospitals, one must first of all be aware that the medical team is not operating in the confined, antiseptic and comfortable context of the hospital. The patient is not coming to it, instead it goes to the patient and his operating environment. The latter is rarely suited for it to work under good conditions of comfort, safety, hygiene, ergonomy.

Currently, it has no working plan or medical care zone to properly prepare the equipment needed to install perfusion or other medical devices, as well as for preparing medicines.

The medical team then “copes” to get the best result from the equipment that is present, that is they place drugs, syringes and other medical devices on the medical bag or case, or even on the ground with the drawbacks of precarious balance and approximate hygiene of the abovementioned supports.

It is then confronted with the following problems:
Space management, because the medical care zone is not always properly delimited around the patient and the equipment is often scattered (risk of loss, substitution or loss of equipment). On the other hand, the medical team can operate in areas that are often restricted, leaving very few opportunities to adapt.
Risk management because the personnel operating around the victim is focused on the latter and not on what is placed directly on the ground, as ampoules of drugs (full or empty), syringes with their needles, catheters containing the patient's blood needed for certain analyses often are.

Hygiene management is more or less random due to the weather (wind that drives dust, rain, snow . . . ), the intervention site (building construction site, unhealthy spaces, public highway . . . ) and the movements of rescue personnel around the patient.
Time management when the environment is “hostile” to medical care, thus forcing the medical team to spread sheets or look for chairs or tables or any means making it possible to place equipment needed for working cleanly. The time lost in this setting up process occurs to the patient's detriment.
Personnel management because it is not unusual to transform rescue personnel into “perfusion stands” when unable to hook the solution to an element in the patient's vicinity. This can be penalizing for organizing medical care, the medical care team having personnel taken away for this purpose.
Light management because the intervention often takes place far from light sources and it is complicated to work in twilight or a dark night.
Waste management because most of the time it has only DASRI (infectious medical waste) bags wherein can be found at the end not only DASRI waste but those which could be disposed of in an ordinary trash can, thus bringing about additional waste treatment costs.

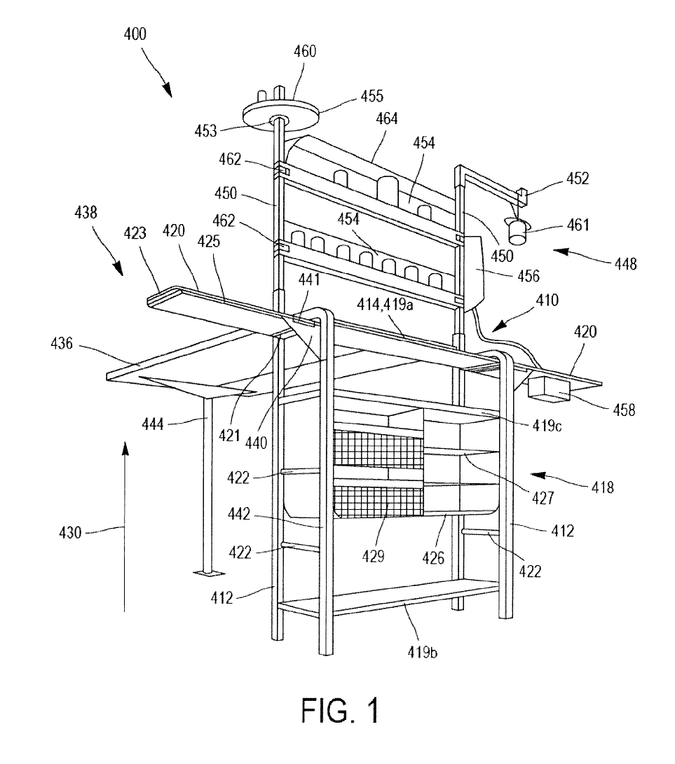

A device having been the subject of a patent application, US2006076861, is known in the prior art. The latter describes a kitchen pack (FIG. 1) comprising a certain number of characteristics which could a priori respond to the problem previously mentioned. This device has as its main disadvantage a bad mutual arrangement of its different features, weight and a space requirement too high both in the storage or transport position and in the operating position, the impossibility of keeping its working surfaces horizontal on rough ground and finally too slow an installation time for use in the extra-hospital medical field, that is under conditions of extreme emergency.

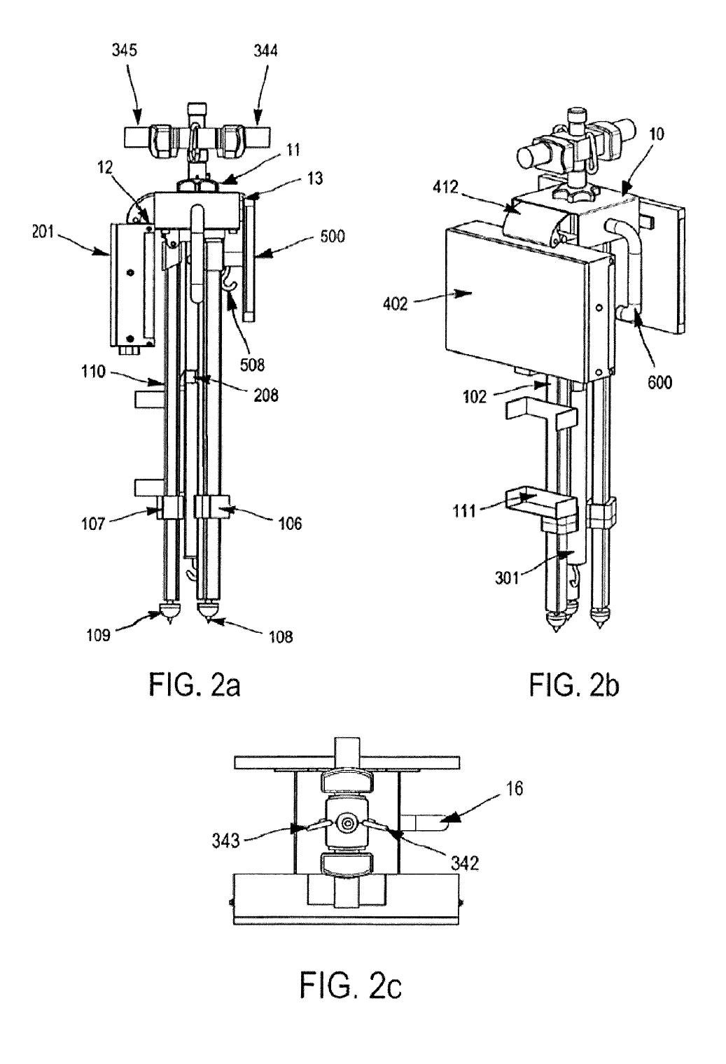

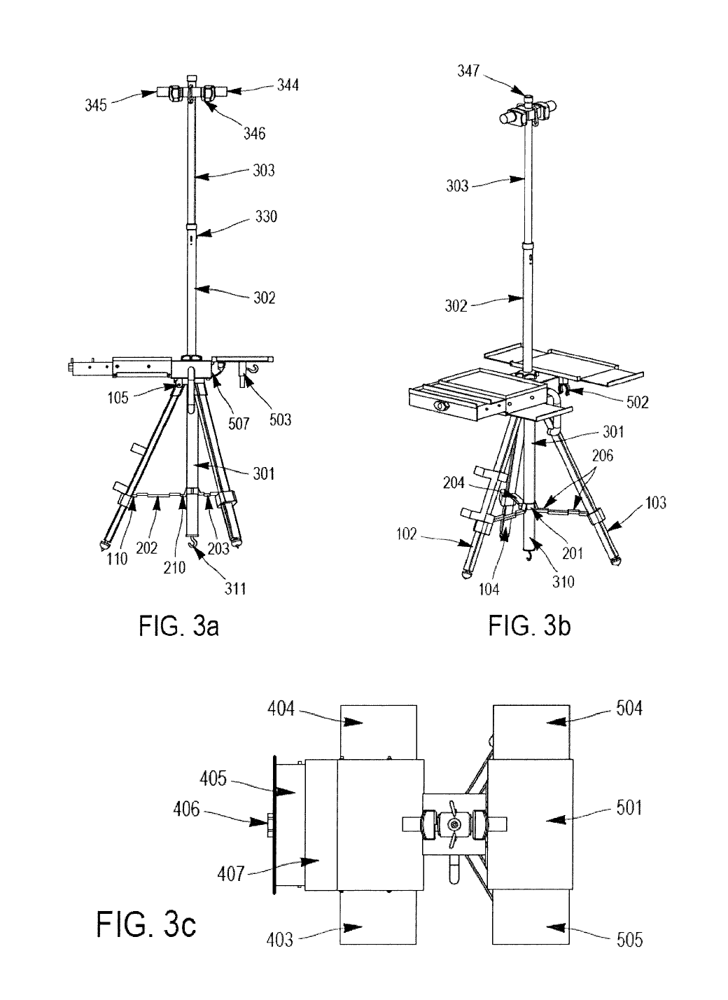

The invention described below and illustrated in FIG. 2 (closed position, for storage or transport) and FIG. 3 (deployed or operating position) makes it possible to apply a concrete solution to the problems previously cited.

This extra-hospital workstation includes:

According to a first feature, a platform having an upper face, a lower face and side faces, connected to:
at least one tray, by pivoting means
possibly a box enclosing a drawer, by pivoting means,
a central mast
folding legs on its lower face
a gripping means to facilitate its transport.

According to a second feature, the tray, provided with rims, includes laterally at least one slide in which is positioned another tray, capable of sliding, as well as at least one gripping means. When the extra-hospital medical workstation is in its operating configuration, it can take a first position in which its upper face is horizontal, a position in which it is functional, and a second position so-called storage (or transport) position where this same face is vertical.

According to a third characteristic, the box enclosing a drawer consists of an upper wall, a lower wall and several side walls. The upper wall of the casing is provided with rims, which constitutes a working tray. The box is attached to the platform at a side wall by pivoting means so that when the extra-hospital medical workstation is in the operating configuration, it can assume a first position in which it is functional and its upper wall is horizontal, and a second position, so-called storage (or transport) position where the same face is vertical.

According to a fourth feature, the box enclosing the drawer includes at least one slide wherein is positioned a second tray, capable of sliding.

According to a fifth feature, the front face of the drawer includes at least one opening making it possible to allow a self-adhesive tape to pass and/or to temporarily store a medical device.

According to a sixth feature, the folding and telescoping legs are in the form of a tripod with a central mast, also telescoping, offering a gripping means in its lower portion as well as at least one lighting means and a gripping means in its upper portion. The latter also offers the possibility of introducing at its upper end a device for protection against bad weather.

According to a seventh feature, the lower portion of the mast is connected through central crosspieces to the three legs. These crosspieces pivot around a horizontal axis attached to the legs as well as around another horizontal axis attached to a ring, itself sliding on the lower portion of the central mast and also capable of fixing itself. These crosspieces laterally offer gripping systems. According to an eighth feature, a gripping system is attached to one of the legs which can accept a needle receptacle.

According to a ninth feature, the gripping means is a handle connected to a side face of the platform.

According to a tenth feature, the structure comprises means of automatic deployment making it possible to deploy jointly the mast, the folding legs and said at least one working tray.

According to an eleventh feature, the automatic deployment means are selected from a list comprising:
a rack-and-pinion system wherein the pivoting means consists of a rack positioned on the mast and at least one pinion attached to said at least one working tray and cooperating with the rack so as to deploy or to fold the tray by sliding the mast inside the platform, and a system of spacers connecting the folding legs to the mast so as to deploy or fold the folding legs by sliding the mast inside the platform;
a cable-pulley system comprising a cable which connects the mast and said at least one working tray, said cable cooperating with a pulley so as to deploy or fold the tray by sliding the mast inside the platform, and a system of spacers connecting the folding legs to the mast so as to deploy or fold the folding legs by sliding the mast inside the platform;
spacer systems connecting said at least one working tray and connecting the folding legs to the mast, so as to deploy or to fold said at least one working tray and the folding legs by sliding the mast inside the platform;
a system of cylinder enabling the deployment of said at least one tray as well as the folding legs by connecting to a reserve of fluid under pressure.

According to a twelfth feature, the structure comprises a raised lighting system.

These features and still more will be clearly revealed by the remainder of the description.

To settle the object of the invention illustrated in a non-limiting manner, it is appropriate to refer to the following figures where:

FIG. 1 is a view of a modular structure according to the prior art.

FIG. 2 shows successively a transverse section view 2a, then a lateral ¾ view, at a slight downward angle 2b and finally a top view 2c of the structure in its storage or transport configuration according to a first embodiment.

FIG. 3 shows successively a transverse section view 3a, then a lateral ¾ view at a slight downward angle 3b and finally a top view 3c of the structure in its operating configuration according to a first embodiment.

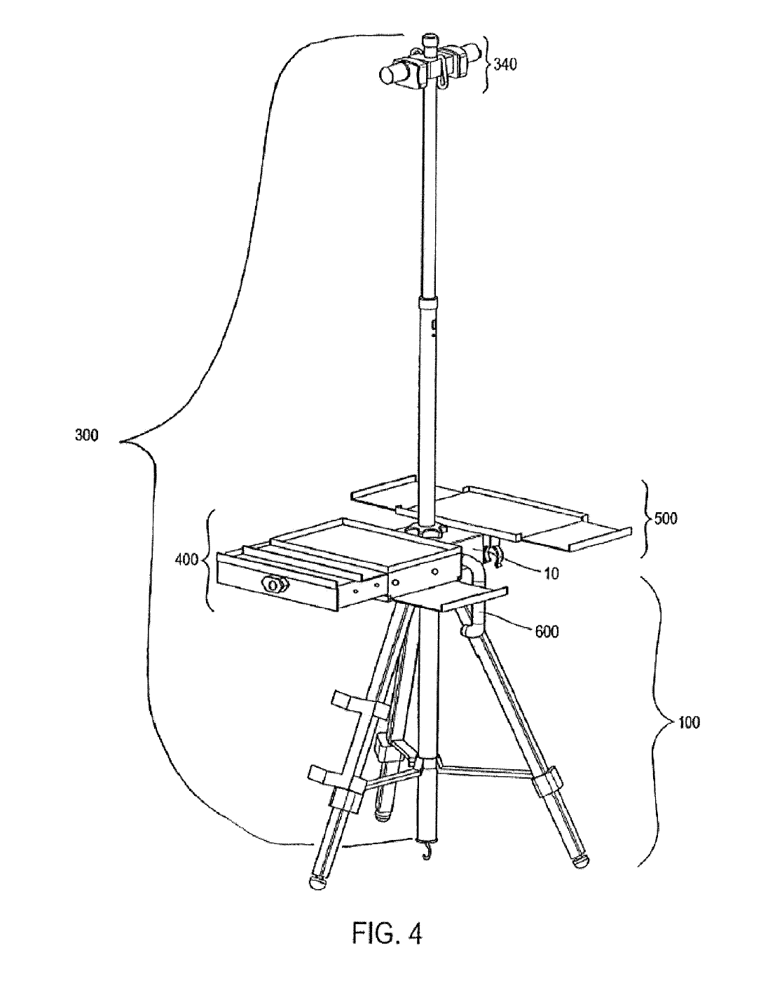

FIG. 4 shows a ¾ side view at a slight downward angle of the structure in its operating configuration according to a first embodiment.

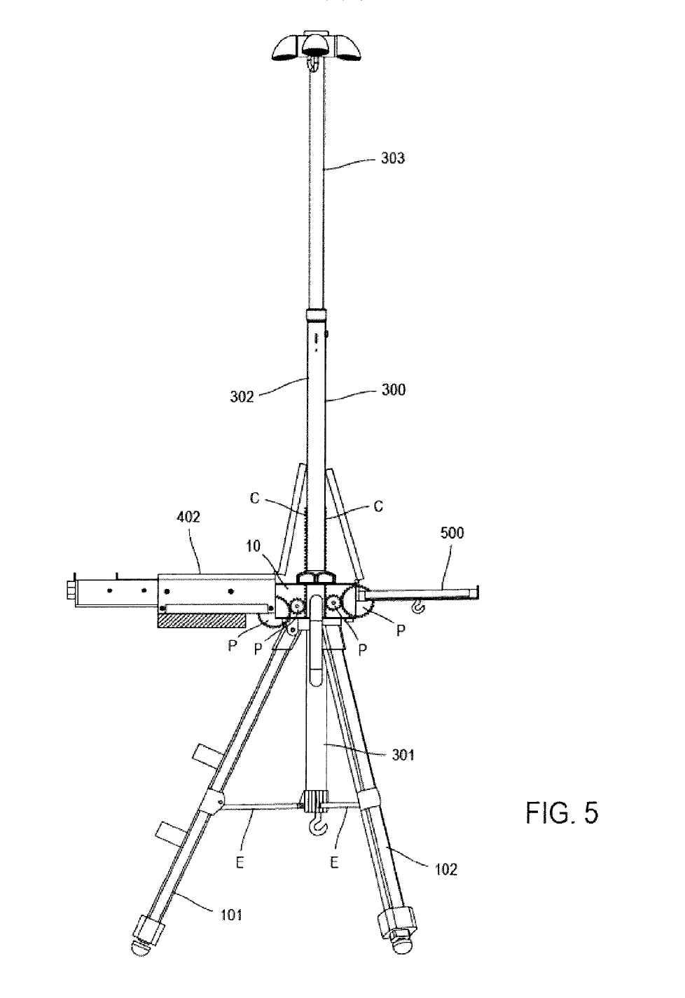

FIG. 5 shows a transverse section view of the structure in its operating configuration according to a second embodiment.

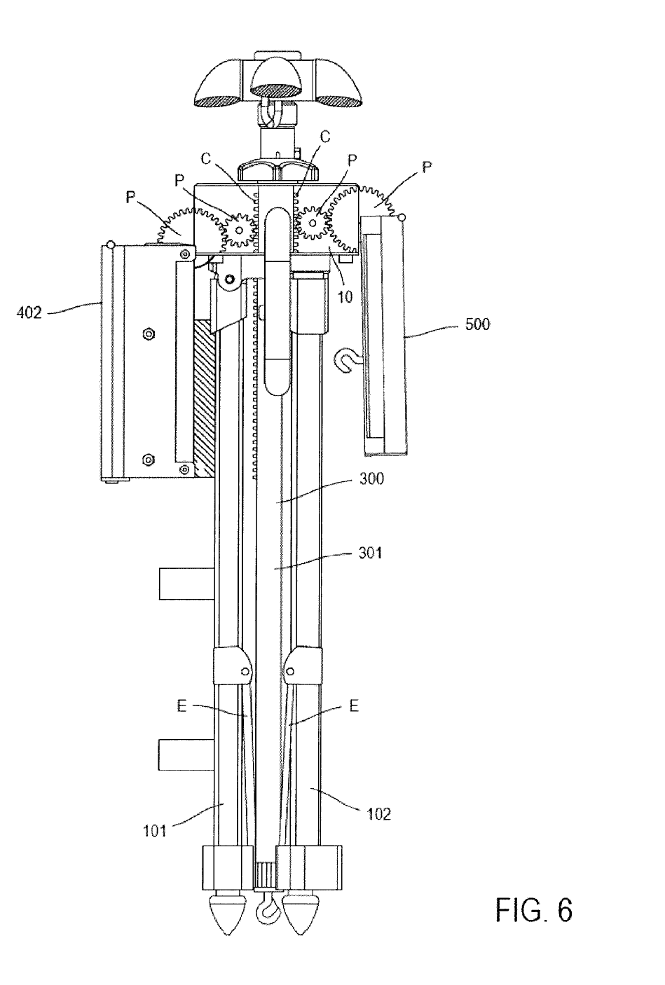

FIG. 6 shows a transverse section view of the structure in its storage or transport configuration according to a second embodiment.

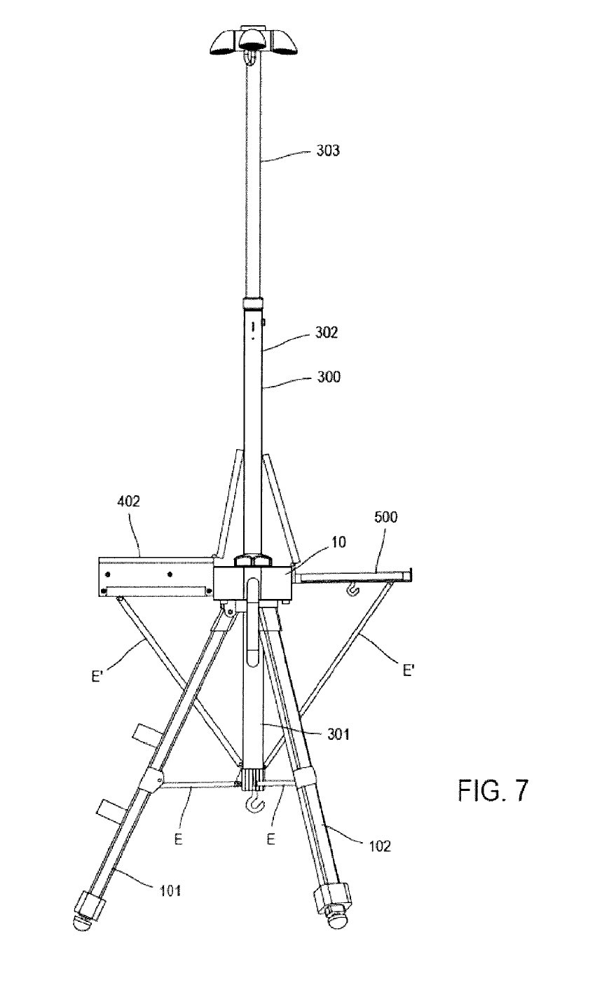

FIG. 7 shows a front view of the structure in its operating configuration according to a third embodiment.

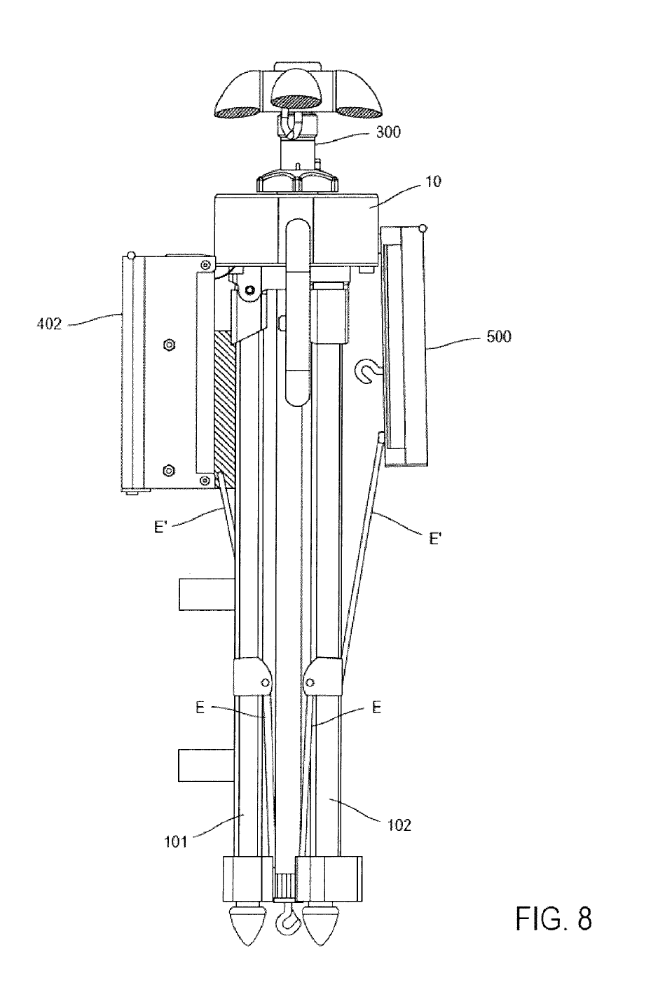

FIG. 8 shows a front view of the structure in its storage or transport configuration according to a third embodiment.

In order to make the object of the invention more concrete, it is now described in a non-limiting manner illustrated in the figures of the drawings.

The support structure according to the invention is successively referred by FIGS. 2, 3 and 4. It comprises a platform or base 10 connected on its lower face with a tripod 100 composed of 3 legs 102, 103, 104 each articulated by a horizontal axis of rotation 105 at their proximal end. Each leg consists of several, three for example, tubes giving it the ability to telescope. The tube with the largest circumference as well as the tube with the next smallest circumference are each provided at their distal end with a lever system 106 and 107 blocking the tube which has the next smallest circumference. According to one variant, the tube with the greatest circumference as well as the tube with the next smallest circumference are each provided at their distal end with a clamping ring instead of the blocking lever system 106 and 107. These clamping rings make it possible to block movement between the tubes. The last tube, the one with the smallest circumference, has its distal end provided with a screw 108 in which is articulated a half-sphere 109 made of rubber making it possible to put the structure into contact with the ground either through the half-sphere (hard ground) or through the sharp end of the screw (loose ground). The leg 102 located below the drawer has an attachment system for a secure container for needles 111. A crosspiece 200 allows the mutual articulation of the three legs 102, 103, 104. It consists of a central ring 201 sliding around the lower portion of the central mast 301 and 3 arms 202, 203, 204 connected at their distal ends to the blocking lever system 106 of the legs with the largest circumference around a horizontal axis of rotation 110 and, in their portions proximal to the ring 201, also around a horizontal axis of rotation 210. Thus, when one of the legs is extended, the two others also open in the same manner without any other action. The central ring 201 is provided with a clamping screw 208 which allow it to be attached to the central mast during its storage or transport, avoiding any untimely opening of the tripod but also, once the structure is deployed, thus conferring on the latter a certain rigidity and stability.

The three arms are equipped, laterally and at each of the two ends, with a clip 204 of the “crocodile” type offering the possibility of attaching a bag to the inside of each of the 3 crosspieces. These bags open automatically following the opening action of one of the three legs.

The central mast 300 consists of several, three for example, tubes which deploy vertically and upward. The tube located in the lower portion 301 having a circumference greater than the other two is integral with the platform, or base, in its proximal portion. Its distal portion is equipped with a fixed ring 310 preventing the movable ring from falling and offering a method of attaching the structure to an anchoring point, in the form of a hook 311 oriented downward.

The intermediate tube has its deployment height adjustable by means of a clamping thumb wheel 11 attached to the upper face of the platform 10.

The last tube 303, the one having the smaller circumference, also has at its proximal end a lug 330 entering into openings provided for this purpose, one being located in the proximal portion and the other being located in the distal portion of the intermediate tube 302, allowing it to have two operating positions, one folded and the other deployed. Its distal end accepts a lighting and gripping module 340. This module offers laterally a fixed gripping system, for a bag of solution, of the snap hook type 342, 343. It also offers an individualized and orientable lighting system 344, 345 of the drawer 400 and the tray 500. The lamps are attached by their lower portion to the base of the module by a cranked articulation with a horizontal axis 346 conferring on it the possibility of maintaining the desired orientation position. Finally, the distal end of this tube makes it possible to accept the introduction of a rod composing the base of a system for protection against bad weather.

The tubes are provided with a vertical rib and a guide preventing any rotation around a vertical axis.

According to one variant, the structure many not include the elevated lighting system while still including a raised gripping system. The drawer module 400 is articulated about a horizontal rotation axis 12 located in the lower rear portion of the box. In the storage configuration, the front face of the drawer is oriented toward the ground and the box is held in this position by the action of a springloaded blade 412 attached to the rear face of the box and clipping itself automatically to the inside of the base 10. To put the box into the operating configuration, it is then necessary to press on this leaf so as to release it, then the drawer box needs to be given a 90° rotary movement upward around a horizontal axis. The drawer has, in its upper wall, a working tray 402 surrounded by rims limiting the falling of elements which would be placed on it. The lower wall of the box shelters two appended trays each provided laterally with a rim. These trays, equipped with stops, slide laterally, one 403 to the right and the other 404 to the left of the box offering the possibility of having small equipment for medical analyses available in particular. These two trays are held in the closed position by the action of a magnet placed inside the side rim of the sliding trays.

The front face of the box shelters the drawer 405 as well as a third sliding tray 407 located under the upper wall of the box but above the drawer. The locking system of the drawer also conditions the locking of this last tray. The drawer slides on two rails with stops, fixed horizontally inside the side walls of the box. The front face of the drawer is equipped with a pushbutton 406 holding the drawer in the closed position, as well as the tray that is on top of it when it is pressed in. To allow opening, it is enough to exert pressure on the latter which will then partly leave its accommodation to offer a gripping means on the drawer. The front face of the drawer also has two vertical slots, one allowing the passage of medical adhesive tape and the other an identifying ribbon for injectable medicine. Finally, it is equipped with an opening making it possible to interpose a catheter with its sheath while awaiting analysis.

The tray 500 is articulated about a horizontal axis of rotation 13. It is held in the storage position (main wall 501 oriented vertically) by means of a half circle 502 in the form of a “springloaded blade” which surrounds the mast. This halfcircle is extended by a cylinder 503 which is attached to the lower wall of the tray. This cylinder is also equipped with a hook 508. To put the tray in the operating position it must exert a force from back to front and from bottom to top while causing it to pivot by 90°, freeing it from the pressure of the halfcircle. Its main wall is then oriented horizontally and it is held in this position by a spring-loaded blade 507 which clips itself automatically. To reposition the tray into the storage configuration, pressing on the springloaded blade releases the blocking mechanism, allowing it to pivot freely. This tray shelters two appended trays provided laterally with a rim. These trays, equipped with stops, slide laterally, one to the right 504 and the other to the left 505 of the main tray, thus granting it the possibility of increasing its surface area. These two trays are held in the closed position by the action of a magnet placed inside the side rim of the sliding trays.

A handle 600, constituting a gripping means to facilitate transport, is attached to a side face of the platform 10.

Finally, an appended lamp is associated with the structure for lighting the patient.

In summary, this extra-hospital medical workstation, very compact in the storage configuration, makes it possible to make available to the State Certified Nurse and/or the doctor, once deployed, two working trays:
The first in the form of a drawer, making it possible to store, among other things, the equipment required for setting up perfusion. It facilitates the preparation of drugs and makes it possible to place equipment for analysis and care.
The second, in the form of an extensible tray designed to position the doctor's intubation and aspiration equipment for example.

Moreover, this tripod makes possible, to the benefit of the medical or paramedical team, the nearly immediate availability of:
Two trash bags (yellow or black for example) and a needle receptacle, attached to the legs of the tripod below the working trays, allowing sorting of waste and the removal of needles with complete safety.
A central telescoping mast which can accept a device for protection from bad weather with, at its peak, an individualized lighting module for the two working trays and the necessary equipment for attaching bags of solution (snap hook type attachment).

This working tool, once made available to the medical team, notably improves intervention work in environments outside the hospital in terms of:
Of managing space clearly identified and organized around the tripod.
Of risk management which has all of the equipment positioned on working trays while leaving nothing dangerous lying on the ground.
Of hygiene management because the equipment is positioned on a clean tray and it is possible to add if needed means of protection against bad weather to protect patient care zones as well as the two working trays.
Of time management because the setting up of the tripod in the operating configuration requires no more than ten seconds even under extreme conditions.
Of personnel management by releasing the “person operating as a perfusion stand” from his support role and reintegrating him into the first aid team.
Of lighting management with the working trays lit to favour the identification and the preparation of drugs and an orientable appended lamp to light the medical care zone.
Of waste management with the presence, as soon as the tripod is opened, of two types of trash bags and of the needle collector.

In conclusion, this compact and light workstation makes it possible to apply a general response pertaining to a process for improving the method of out-of-hospital care while approaching as closely as possible hospital requirements.

According to other possible embodiments, such as those shown in FIGS. 5 through 8, the structure can comprise automatic deployment means making it possible to deploy jointly the folding legs 102, 103, 104 as well as the working trays 402 and 500 using a reduced number of actions by the user, so as to reduce the time required to change the structure from its transport or storage position to is operating position. This time saving is particularly important in out-of-hospital interventions because in the event of an emergency (for example in the event of a cardiac arrest, or very serious bleeding), each second saved can have a determining impact. According to the variables described below, the structure comprises automatic deployment means making it possible to jointly deploy the mast 300, the folding legs 102, 103, 104 and the working trays 402 and 500.

As illustrated in FIGS. 5 and 6, according to a second embodiment, the automatic deployment means comprise on the one hand a rack-and-pinion system allowing deployment of the working trays 402 and 500, and on the other hand a system of spacers E making possible to deploy the folding legs 102, 103, 104 and the mast 300 can slide inside the platform 10.

The rack-and-pinion system comprises, to articulate each of the working trays 402 and 500, a rack C positioned on the mast 300, more precisely on the tube located in its lower portion 301, and two pinions P of which a first pinion P is positioned in the platform 10 and engages with the rack, and of which a second pinion P is attached to a working tray 402 and 500 and meshes with the first pinion. This rack-and-pinion system thus forms a means for pivoting the working trays 402, 500 with respect to the platform 10.

Thus, the mast 300 being able to slide inside the platform 10 when the user deploys the mast 300 and pulls upward on it, the rack C drives the pinions P in rotation (more precisely the rack C drives the first pinion which in its turn dries the second pinion), said pinions P thus driving the deployment of the working plates 402 and 500 with respect to the platform 10. Reciprocally, when a user wants to put the structure away, he folds the mast 300 by pressing downward on it, the rack C drives the pinions P in rotation, and the working trays 402 and 500 are folded.

The automatic deployment of the folding legs 102, 103 and 104 for its part is ensured by a system of spacers E. Each folding leg 102, 103, 104 is connected to one spacer E using a pivot linkage with an axis orthogonal to both the folding leg 102, 103, 104 and to the spacer E. Each of the spacers E being attached to the mast 300, and more precisely to the distal end of the tube located in the lower portion 301 using a pivot linkage with an axis orthogonal to both the mast 300 and to the spacer thus linked. The axes of the pivot linkages of each spacer E are parallel to the axis of rotation of the connection between the folding let 102, 103, 104 to which the spacer E is connected and the platform 10.

This system of spacers E thus makes possible, when the user deploys the mast 300 and pulls upward on it, to deploy the folding legs 102, 103, 104 by sliding the mast 300 inside the platform 10 by deploying the spacers E.

Reciprocally, when a user wants to put the structure away, he folds the mast 300 by pressing downward on it, the spacers E fold against the mast 300, thus folding the folding legs 102, 103 and 104.

This embodiment therefore allows deploying or folding the working trays 402, 500 and the folding legs 102, 103, 104 jointly in a single movement of the user, who pulls on or pushes the mast 300.

As illustrated in FIGS. 7 and 8, according to a third embodiment, the automatic deployment means comprise on the one hand a system of spacers E′ making it possible to deploy the working trays 402 and 500, and on the other hand a system of spacers E making it possible to deploy the folding legs 102, 103, 104 similarly to the spacer system E of the second embodiment, the mast 300 sliding inside the platform 10.

The spacer system E′ therefore allows deployment of the working trays 402 and 500 with an operation equivalent to the spacer system E allowing deployment of the folding legs 102, 103, 104 of the second embodiment. The spacer system E′ allowing deployment of the working trays 402 and 500 therefore comprises two spacers E′, each being attached both to a working tray 402 or 500, and to the mast 300, and more particularly to the distal end of the tube located in the lower portion 301. The spacers E′ are each attached to a working tray 402 or 500 and to the mast 300 according to a pivot linkage with an axis parallel to the axis of rotation of the linkage between the working tray 402 or 500 to which the spacer E′ is connected and the platform 10.

This spacer system E′ thus allows, when the user deploys the mast 300 and pulls upward on it, to move the working trays 402 and 500 by sliding the mast 300 inside the platform 10 while deploying the spacers E′.

Reciprocally, when a user wants to put the structure away, he folds the mast 300 by pressing downward on it, the spacers E′ fold against the mast 300, thus folding the working trays 402 and 500.

According to a fourth embodiment, the automatic deployment means comprise on the one hand of a cable-pulley system allowing deployment of the working trays 402 and 500, and on the other hand of a system of spacers E allowing deployment of the folding legs 102, 103, 104 similarly to the spacer system E of the second embodiment, the mast 300 sliding inside the platform 10.

The cable-pulley system comprise cables each of which connects a tray 402 or 500 to the mast 300, and pulleys each attached inside the platform 10 opposite a working tray 402 or 500, each cable passing through a pulley. The cables are connected to the intermediate tube 302, so that when the mast 300 is unfolded, the cables are tightened and pull the working trays 402 and 500 so as to deploy them.

The pulleys allow a reduction in the space requirements of the cables when they are under tension, so that they do not disturb the work of the users.

When a user wants to put the structure away, he folds the mast 300 by pressing downward on it, the cables loosen and the working trays 402 and 500 fold under the influence of gravity.

According to a fifth embodiment, the automatic deployment means comprise a system of cylinders which connect each of the working trays 402 and 500 with either the platform 10 or the mast 300, and a system of cylinders which connect each of the folding legs 102, 103, 104 to the mast 300.

These cylinders are positioned in such a manner that when they are in the “extended” position the working trays 402, 500 and the folding legs 102, 103, 104 are deployed, and when said rams are in the “retracted” position the working trays 402, 500 and the folding legs 102, 103, 104 are folded.

The cylinders can be electric actuators, so that they can be controlled by a switch, the structure then comprising an electric battery allowing the cylinders to be supplied with electrical energy.

The cylinders can also be gas cylinders, which deploy when they are connected to a reserve of gas under pressure. For example, all the cylinders can be connected to the same gas circuit which can be supplied with gas under pressure by connecting a gas bottle to a nozzle, thus providing an entry to the gas circuit. The user would then only have to connect a bottle comprising gas under pressure to the nozzle for the cylinders to go into the “extended” position. According to a variant of this embodiment, a bottle of gas under pressure is connected to the cylinder when the structure is in the storage position, so that the gas inside the cylinders is under pressure, the cylinders being retained in the “retracted” position by blocking means, such as pins positioned over the cylinders' path. During the use of the structure, the user releases the pins from the blocking means, for example by operating a pushbutton connected to a spring mechanism causing the pins to leave the cylinders' path. The cylinders thus released from the blocking mechanism and being full of gas under pressure, they deploy and assume the “extended” position, thus deploying the folding legs 102, 103, 104 and the working trays 402, 500. According to still another variant, a hydraulic valve is interposed between a reserve of gas under pressure and the cylinders and the hydraulic valve is automatically switched to drive the cylinders in extension and thus ensure the deployment of the complete structure of the system conforming to the invention.

According to another possible variant of this embodiment, the mast 300 can also be deployed by a cylinder. In the case where the cylinders are gas cylinders, the cylinder deploying the mast 300 can also be connected to the same gas circuit as the other cylinders. The various embodiments presented are only examples of implementing the invention. Thus, they can easily be combined, for example by combining the various systems making it possible to automatically deploy the working trays 402, 500 and the folding legs 102, 103, 104.

Moreover, the invention is not strictly limited to the particular implementation of the means which are described, but extend to all variant conforming with its spirit and with all equivalent means, that is those fulfilling the same function as the means described.

Finally, according to a variant of all the embodiments previously described, the working trays 402 and 500 comprise a cover, articulated for example with said working trays 402 and 500 using a pivoting linkage.

The fact that said working trays 402 and 500 comprise a cover allow them to be protected against various dirt which can be deposited during storage or transport of the structure. In addition, the covers can also serve for storing equipment by being designed with thickness so as to form a cavity wherein it is possible to store equipment, or even with attachment means allowing the attachment of equipment needing to be rapidly available during interventions. The equipment thus stored can be stored either inside the cover, being protected from dirt when the cover is closed, or even outside the cover so as to be available even if the structure is still in the storage or transport position. These two variants of the cover can obviously also be combined.

1. A structure for medical equipment, intended to be used primarily by medical or paramedical personnel in an extra-hospital environment, comprising at least one working tray as well as a raised attachment system and fixing means allowing the attachment of accessories for storing medical waste, wherein said structure further comprises a platform having an upper face, a lower face and several side faces, the platform being connected at its lower face to folding legs and, by a pivoting means to at least one working tray, and in that said platform has a mast passing through it, and in that it includes a gripping means, all of it to allow rapid, stable and to standard transport and implementation of the assembly of these different elements, both on flat ground and on broken ground.

2. The structure according to claim 1, wherein a working tray provided with rims constitutes the upper wall of a box enclosing a drawer, that said box consists of an upper wall, a lower wall and several side walls and is attached to the platform at a side wall by a pivoting means so that when the extra-hospital medical workstation is in the operating configuration, it can assume a first position in which it is functional and its upper wall is horizontal, and a second position so-called storage (or transport) position where this same face is vertical.

3. The structure according to claim 2, wherein the box enclosing the drawer includes at least one slide wherein is positioned a second tray capable of sliding.

4. The structure according to claim 1, wherein it comprises automatic deployment means allowing joint deployment of the folding legs and said at least one working tray.

5. The structure according to claim 4, wherein the automatic deployment means are selected from the list comprising:
a rack-and-pinion system wherein the pivoting means consists of a rack positioned on the mast and at least one pinion attached to said at least one working tray and cooperating with the rack so as to deploy or to fold the working tray by sliding the mast inside the platform, and a system of spacers connecting the folding legs to the mast so as to deploy or to fold the folding legs by sliding the mast inside the platform;
a cable-pulley system comprising a cable which connects the mast and said at least one working tray, said cable cooperating with a pulley so as to deploy or fold the working tray by sliding the mast inside the platform, and a system of spacers connecting the folding legs to the mast so as to deploy or to fold the folding legs by sliding the mast inside the platform;
spacer systems connecting said at least one working tray and connecting the folding legs to the mast so as to deploy or to fold said at least one working tray and the folding legs by sliding the mast inside the platform;
a system of cylinders allowing deployment of said at least one working tray as well as the folding legs by connecting to a reserve of fluid under pressure.

6. The structure according to claim 1, wherein the plate, provided with rims, includes laterally at least one slide wherein is positioned a second tray, capable of sliding, as well as at least one gripping means so that, when the extra-hospital medical workstation is in the operating configuration, it can take on a first position where its upper face is horizontal, a position in which it is functional, and a second position so-called storage (or transport) position where the same face is vertical.

7. The structure according to claim 1, wherein the folding and telescoping legs are in the form of a tripod provided with a central mast, also telescoping, offering gripping means on its lower portion as well as lighting means and a gripping means on its upper portion, also allowing the possibility of introducing at its upper end a device for protection against bad weather.

8. The structure according to claim 7, wherein the lower portion of the mast is connected through central crosspieces to the three legs, that said crosspieces pivot around a horizontal axis attached to the legs as well as around another horizontal axis attached to a ring, sliding in its turn on the lower portion of the central mast and also being able to attach itself and that said crosspieces offer gripping systems laterally.

9. The structure according to claim 3, wherein the trays capable of sliding laterally are accommodated in the lower wall of the box and that the tray capable of sliding front to back is accommodated between the upper wall of the box and the drawer.

10. The structure according to claim 2, wherein the front face of the drawer is oriented downward in the storage position and that it includes at least one opening allowing a self-adhesive tape to pass through and/or to store temporarily a medical device.

11. The structure according to claim 1, wherein it comprises a raised lighting system.

12. The structure according to claim 1, wherein the gripping means is a handle connected to a side face of the platform.

13. The structure according to claim 1, wherein on one of the folding legs is attached a gripping system which can accept a needle receptacle.

14. The structure according to claim 1, wherein the working trays comprise a cover.
